(19) United States
(12) Patent Application Publication
(10) Pub. No.: US 2024/0260899 A1
(43) Pub. Date: Aug. 8, 2024

(54) BIOMIMETIC, NANOFIBER-BASED AND DIRECTIONAL MOISTURE-WICKING ELECTRONIC SKINS AND FABRICATION METHODS THEREOF

(71) Applicant: City University of Hong Kong, Hong Kong (HK)

(72) Inventors: Jinlian HU, Hong Kong (HK); Chuanwei ZHI, Hong Kong (HK); Shuo SHI, Hong Kong (HK); Yifan SI, Hong Kong (HK)

(21) Appl. No.: 18/412,641

(22) Filed: Jan. 15, 2024

Related U.S. Application Data
(60) Provisional application No. 63/482,799, filed on Feb. 2, 2023.

(51) Int. Cl. A61B 5/00; A41D 1/00; A61B 5/024; A61B 5/0245; A61B 5/256; A61B 5/28; D04H 1/43; D04H 1/4318; D04H 1/4382; D04H 1/56; D04H 1/728; D06M 11/74; D06M 101/22

(52) U.S. Cl. CPC A61B 5/6804; A41D 1/002; A61B 5/256; D04H 1/43; D04H 1/4318; D04H 1/43838; D04H 1/56; D04H 1/728; D06M 11/74; A41D 2400/00; A41D 2500/30; A61B 5/02438; A61B 5/0245; A61B 5/28; A61B 2562/0285; D06M 2101/22; D06M 2200/00; D10B 2401/021; D10B 2401/022; D10B 2501/00

(57) ABSTRACT

A nanofiber-based directional moisture wicking electronic fabric and preparation methods thereof are provided. The electronic fabric includes hydrophobic fibrous layer, hydrophilic fibrous layer, and conductive functional coating layer. In the preparation method of the nanofiber-based electronic fabric, the hydrophobic nanofibers, the conductive coating layer, and the hydrophilic nanofibers are successively constructed by the combination of electrospinning and electrostatic spraying technology. Through the construction of hydrophilic and hydrophobic differences, the all-fibrous electronic fabric of the invention is enabled to transport sweat from the skin surface to hydrophilic nanofibers to maintain good comfort and has the merit of good electricity at the same time. The all-fibrous electronic fabric is easy to be fabricated and has a wide application prospect in the fields of waterproof and moisture permeable clothing and intelligent wearable electronics.

### CROSS-REFERENCE TO RELATED APPLICATIONS

The present application claims priority from U.S. provisional patent application Ser. No. 63/482,799 filed Feb. 2, 2023, and the disclosure of which is incorporated herein by reference in its entirety.

### FIELD OF THE INVENTION

The present invention generally relates to the technical field of electrospinning. More specifically the present invention relates to a biomimetic, nanofiber-based and directional moisture-wicking electronic skin and a fabrication method thereof.

### BACKGROUND OF THE INVENTION

The global interest in wearable bioelectronics, capable of detecting and quantifying physiological data arising from human movements, has surged. Conventional rigid electrodes, typically affixed to the skin or organs using tapes or clips, often yield incompatible contact with the human body, leading to distorted or highly noisy physical signals. Recent advancements in flexible electronic skins address these issues, offering characteristics such as lightweight, ultra-thinness, and comfort. These innovations find applications in personalized health management, man-machine interfaces, and artificial intelligence. However, many flexible bioelectronics with impressive acquisition performance are constructed using impermeable polymer membranes or gels, impeding gas or moisture exchange between the human skin and the ambient environment. This limitation can result in interference from externally induced thermal or moisture effects. Extended contact periods can lead to sweat deposition, causing discomfort and signal distortion.

Electrospinning technology has rapidly evolved for large-scale manufacturing of fibrous membranes. Nanofiber networks created through electrospinning exhibit a high specific surface area, excellent flexibility, and diverse pore sizes, making them promising candidates for highly sensitive and breathable electronic skins. Recent studies have focused on developing high-performance, monofunctional pressure sensors or nanogenerators using nanofibrous membranes, demonstrating good conductivity or energy harvesting in specific applications.

However, the development of electrospun fibrous membranes has predominantly focused on monotonically hydrophilic or hydrophobic architectures, resulting in skin humidity and limited moisture transfer between the body and electronics. This can lead to skin discomfort and potential inflammation. In the context of large-scale practical applications of electronic skins, factors such as versatility, wearable comfort, and energy-saving performance are crucial.

MXene, a novel 2D material derived from the chemical exfoliation of bulk MAX crystalline structures, has garnered attention. Represented by the formula Mₙ₊₁XₙTx (n=1, 2, or 3), where M denotes transition metals (e.g., Ti, Cr, and V), X denotes carbon and/or nitrogen, and Tx represents surface functional groups (hydroxyl, oxygen, or fluorine terminals), MXene exhibits excellent metallic conductivity, thermal conductivity, and a high surface area. Various electronic skins composed of Ti₃C₂Tx and their hybrids have been designed to achieve piezoresistive sensing properties, including low detection limits, high sensitivity, and long stability. Challenges arise, however, due to the exceptionally high conductivity and interlayer restacking resulting from hydrogen bonding and van der Waals forces, leading to minimal changes in the conductive path under low pressure and low sensitivity. Furthermore, the low mechanical strength compromises long-term deformation and structural integrity under high pressure. Recent efforts have focused on constructing stable interfaces in different MXene-based hybrid nano-/micro-structures through the design of heterogeneous interface interactions. Nevertheless, traditional electronic skins and biosensors based on MXenes are typically fabricated using airproof composite membranes, potentially resulting in uncomfortable wear, microorganism breeding, and skin inflammation.

Therefore, developing moisture-permeable multifunctional electronic skins for physiological monitoring and biomechanical energy harvesting in a single device is of great importance, and the present invention provides a biomimetic, nanofiber-based and directional moisture-wicking electronic fabric/skin to address the need of human-compatible wearable electronic fibres/skins.

### SUMMARY OF THE INVENTION

The present invention provides a biomimetic, nanofiber-based and directional moisture-wicking wearable electronic fabric having an asymmetric heterostructure, and fabrication methods and applications thereof.

In accordance with a first aspect of the present invention, a biomimetic, nanofiber-based and directional moisture-wicking wearable electronic fabric is provided. The electronic fabric includes a hydrophobic nanofiber layer, a superhydrophilic nanofiber layer and a conductive layer. Particularly, the conductive layer is located between the hydrophobic nanofiber layer and the superhydrophilic nanofiber layer, forming a multilayer composite structure so as to realize the conductive and electronic sensing function and generate a surface energy gradient and a push-pull effect to guide moisture unidirectionally. Moreover, the biomimetic, nanofiber-based and directional moisture-wicking wearable electronic fabric has a pressure sensing sensitivity of 75 to 550 kPa⁻¹ in a pressure range from 0 to 20 kPa and a water vapor transfer rate between 12-18 kg·m⁻²·d⁻¹ at 25° C.

When a water drop contacted the hydrophobic PVDF layer, the water was pumped over the hydrophobic side and wetted the superhydrophilic PAN nanofibers side in about 18 seconds. In contrast, when the electronic fabric was flipped and the water droplet contacted the superhydrophilic nanofibrous PAN layer, the water droplet was blocked and spread over the superhydrophilic nanofibers without penetrating the hydrophobic PVDF nanofibers.

In accordance with one embodiment of the present invention, the conductive layer is coated on a side either of the hydrophobic nanofiber layer or the superhydrophilic nanofiber layer.

In accordance with one embodiment of the present invention, the hydrophobic nanofiber layer and the superhydrophilic nanofiber layer are electrospun nanofiber layers.

In accordance with another embodiment of the present invention, t the conductive layer is an electrosprayed conductive layer.

In accordance with one embodiment of the present invention, the hydrophobic nanofiber layer includes one or more high molecular weight hydrophobic polymers selected from polyvinylidene difluoride, copolymers of polyvinylidene difluoride, polyurethane, or polycaprolactone.

In accordance with one embodiment of the present invention, the superhydrophilic nanofiber layer includes one or more high molecular weight hydrophilic polymer selected from polyacrylonitrile, polyvinyl alcohol, nylon or polyethylene glycol.

In accordance with one embodiment of the present invention, the conductive coating layer includes an ultrafine and conductive functional material selected from one or more graphene oxide, titanium carbide, carbon nanotube, carbon black or acetylene black and a metal salt selected from lithium chloride or sodium chloride.

In accordance with one embodiment of the present invention, the size of the ultrafine and conductive functional material is 0.1 to 10 μm.

In accordance with a second aspect of the present invention, a method of fabricating the biomimetic, nanofiber-based and directional moisture-wicking wearable electronic fabric is provided. Particularly, the method including the following steps:
forming either one of the hydrophobic nanofiber layer and the superhydrophilic layer by electrospinning;
electrospraying the conductive coating layer on a side of the hydrophobic or the superhydrophilic layer;
forming the other one of the hydrophobic nanofiber layer and the superhydrophilic layer on the conductive coating layer to form the multilayer composite structure with the conductive coating layer as the middle layer; and drying them to obtain the biomimetic, nanofiber-based and directional moisture-wicking wearable electronic fabric.

In accordance with one embodiment of the present invention, the electrospraying of the conductive coating layer includes adding one or more ultrafine and conductive functional materials into a solution of water and ethanol with an addition of a surfactant to form an electrostatic spraying ink; and electrospraying the electrostatic spraying ink on the side of the hydrophobic nanofiber layer or the hydrophilic nanofiber layer to form the conductive coating layer.

In accordance with one embodiment of the present invention, the solution of water and ethanol has a mass ratio of (5-10):(10-20) of water and ethanol.

In accordance with one embodiment of the present invention, the surfactant is a low molecular weight polyvinylpyrrolidone with an addition amount of 0-5 wt %.

In accordance with one embodiment of the present invention, the ultrafine and conductive functional material selected from one or of graphene oxide, titanium carbide, carbon nanotube, carbon black or acetylene black; and the metal salt selected from lithium chloride or sodium chloride

In accordance with one embodiment of the present invention, the mass ratio among the one or more ultrafine and conductive functional materials, the surfactant and the solution is (2-5):(0-1):20.

In accordance with one embodiment of the present invention, the electrostatic spraying process is conducted by a single-needle electrospraying device with electrostatic spraying conditions of a voltage ranging from 18 kV to 25 kV, an injection pump flow rate of 0.04 mm/min to 0.1 mm/min, and a receiving distance between 5 cm to 15 cm.

In accordance with one embodiment of the present invention, the formation of the hydrophobic nanofiber layer includes adding a hydrophobic polymer and carboxylic carbon tubes to a dimethylformamide and/or acetone solvent and stirring them to obtain a first spinning solution; and electrospinning the first spinning solution to obtain the hydrophobic nanofiber layer.

In accordance with one embodiment of the present invention, the hydrophobic polymer is selected from polyvinylidene difluoride, copolymers of polyvinylidene difluoride, polyurethane, or polycaprolactone.

In accordance with one embodiment of the present invention, the formation of the superhydrophilic nanofiber layer includes adding a hydrophilic polymer and a surfactant to a dimethylformamide solvent and stirring them to obtain a second spinning solution; and electrospinning the second spinning solution to obtain the superhydrophilic nanofiber layer.

In accordance with one embodiment of the present invention, the hydrophilic polymer is selected from polyacrylonitrile, polyvinyl alcohol, nylon or polyethylene glycol.

In accordance with one embodiment of the present invention, the surfactant is selected from sodium dodecyl sulfonate, sodium dodecyl benzene sulfonate or hexadecyl trimethyl ammonium bromide.

In accordance with one embodiment of the present invention, the electrospinning is characterized by using a single-needle spinning device with electrospinning conditions comprising a voltage of 20 kV to 25 kV, a flow rate of syringe pump of 0.03 mm/min to 0.06 mm/min, and a receiving distance of 10 cm to 15 cm.

In accordance with a third aspect of the present invention, a biomimetic, nanofiber-based and directional moisture-wicking electronic skin is provided. Specifically, the electronic skin includes the aforementioned biomimetic, nanofiber-based and directional moisture-wicking wearable electronic fabric with the hydrophobic nanofiber layer contacting human skin.

In accordance with a fourth aspect of the present invention, a single-electrode triboelectric nanogenerator, including the aforementioned biomimetic, nanofiber-based and directional moisture-wicking wearable electronic fabric and one or more electrodes positioned on the conductive layer, is provided. It is worth noting that the single-electrode triboelectric nanogenerator has a response time between 20 to 30 milliseconds, a recovery time between 30 to 40 milliseconds and an areal power density ranging from 0 to 21.6 μW m⁻².

### BRIEF DESCRIPTION OF THE DRAWINGS

Embodiments of the invention are described in more details hereinafter with reference to the drawings, in which:

FIGS. 1A-1L show the material characterization of the biomimetic, nanofiber-based and directional moisture-wicking wearable electronic fabric, in which FIG. 1A shows the SEM image of C-PVDF nanofibers layer, FIG. 1B exhibits the SEM image of C-PVDF/MXene-CNTs layer with an enlarged image as an inset,

FIG. 1C demonstrates the SEM image of PAN nanofibers layer, FIG. 1D shows the diameter distribution of the C-PVDF nanofibers and PAN nanofibers, FIG. 1E illustrates FT-IR measurement of the wearable electronic fabric, FIG. 1F shows the FT-IR measurement of the C-PVDF with different time of electrospraying MXene/CNTs, FIG. 1G displays the XRD patterns of the wearable electronic fabric,

FIG. 1H shows the XPS survey scan of the C-PVDF and C-PVDF/MXene-CNTs,

FIG. 1I depicts O 1s XPS scan spectra of the C-PVDF and C-PVDF/MXene-CNTs,

FIG. 1J displays F 1s XPS scan spectra of the C-PVDF and C-PVDF/MXene-CNTs,

FIG. 1K shows the tensile strength curve of the wearable electronic fabric, and FIG. 1L shows the water vapor transfer rate (WVT) of the wearable electronic fabric;

FIGS. 2A-2H show the directional water transport properties of the biomimetic, nanofiber-based and directional moisture-wicking wearable electronic fabric, in which FIG. 2A is a pie chart showing that water contact angle change with time increasing, FIG. 2B is a schematic diagram showing the change of water contact angle with time increasing, FIG. 2C depicts optical photographs of apparent contact angle change on the hydrophobic C-PVDF nanofibers, FIG. 2D depicts optical photographs of apparent contact angle change on the hydrophilic polyacrylonitrile (PAN) nanofibers, FIG. 2E and FIG. 2F respectively depict the proposed directional water transport mechanism of the electronic fabric from the hydrophobic and hydrophilic layers, and FIG. 2G and FIG. 2H respectively demonstrate the proposed analysis model of the implementation of the directional water transfer in two situations;

FIG. 3A-3H depict pressure sensing performance and the sensing mechanism of the wearable electronic fabric, in which FIG. 3A schematically illustrate the electronic fabric as pressure sensors, FIG. 3B exhibits the sensitivity curve of the electronic fabric with different electrospraying time, FIG. 3C shows the current response curves of the electronic fabric under serial pressures, FIG. 3D displays I-V curves of the electronic fabric under serial pressures, FIG. 3E shows the response and recovery time of the electronic fabric, FIG. 3F demonstrates the detection limit of the electronic fabric, FIG. 3G shows the comparison of the maximum sensitivity and maximum sensing range with prior arts, and FIG. 3H schematically illustrates the sensing mechanism of the electronic fabric;

FIGS. 4A-4G depict performance of the wearable electronic fabric as single-electrode triboelectric nanogenerator (STENG), FIG. 4A schematically shows the working principle of the electronic fabric under triboelectric sensing, FIG. 4B shows the numerical calculations of the potential distribution of the electronic fabric at the contact and separation states, FIG. 4C shows the V_{oc} of the triboelectric nanogenerator (TENG) based on the electronic fabric under different forces, FIG. 4D shows the I_{sc} of the TENG based on the electronic fabric under different forces, FIG. 4E shows the Q_{sc} of the TENG based on the electronic fabric under different forces, FIG. 4F shows the voltage and current, and areal power density of the electronic fabric at various external resistance loadings, and FIG. 4G shows the cycling performance of continuous 5,000 cycles working of the electronic fabric;

FIGS. 5A-5H depict that the wearable electronic fabric is suitable for all-range health monitoring, in which FIG. 5A depict a gait sensing scheme of the electronic fabric, FIG. 5B demonstrates the gait sensing signal and enlarged signal of the electronic fabric, FIG. 5C shows the voice signal of the different words, FIG. 5D depicts the voice signal of the sentence of continuous words speaking, FIG. 5E displays the wrist pulse signal of a 28-year-old male before and after exercise, FIG. 5F shows two sets of wrist pulse signal from two users, FIG. 5G schematically illustrates the signal acquisition and analysis system including signal acquisition, processing, wireless transmission, and mobile application, and FIG. 5H shows the optical and infrared camera images of the electronic fabric on skin before and after running exercise;

FIG. 6 depicts the transmission electron microscope (TEM) image of Ti₃C₂Tx nanosheets;

FIG. 7 depicts the TEM image of the MXene/CNTs ink;

FIG. 8 depicts the scanning electron microscope (SEM) image of sample-1 and sample-2 with spraying time of 2h and 6h;

FIG. 9 shows the surface resistance changes of the DMWES in different samples;

FIGS. 10A-10B depict the mean pore diameters in different layers, in which FIG. 10A shows the mean pore size of PAN layer and FIG. 10B shows the mean pore size of C-PVDF layer;

FIGS. 11A-11B respectively depict the C 1s XPS scan spectra (FIG. 11A) and Ti 2P XPS scan spectra (FIG. 11B) of the C-PVDF and C-PVDF/MXene-CNTs;

FIG. 12 shows the cross-sectional image of the wearable electronic fabric;

FIGS. 13A-13B depict that the thickness of the C-PVDF layer affects the water droplet transfer time, in which FIG. 13A shows the water droplet transfer time while the thickness of the C-PVDF is increased to 18 μm and FIG. 13B shows the water droplet transfer time while the thickness of the C-PVDF is increased to 30 μm;

FIG. 14 depict a PAN nanofibrous membrane sputtered with an Au electrode;

FIG. 15 depicts the results of continuous compression and separation test at 10 kPa;

FIG. 16 is a schematic diagram showing the equivalent circuit of the resistance disposed on the wearable electronic fabric;

FIG. 17 shows that the triboelectric nanogenerator's ability of restoring charge output after water spraying;

FIG. 18 shows the signal detection at joint motions of the wearable electronic fabric;

FIG. 19 depicts the breathing detection ability of the wearable electronic fabric;

FIGS. 20A-20B show the wearable electronic fabric's ability of detecting pulse signals in different environments, in which FIG. 20A shows the pulse signal detection in dry state and FIG. 20B depicts the pulse signal detection in wet state; and

FIG. 21 shows the ECG pulse signals recorded and detected by the wearable electronic fabric.

### DETAILED DESCRIPTION

In the following description, methods for fabricating biomimetic, nanofiber-based and directional moisture-wicking wearable electronic fabric, applications thereof and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In accordance with a first aspect of the present invention, the present invention provides a biomimetic, nanofiber-based, and directional moisture-wicking wearable electronic fabric featuring an asymmetric heterostructure. The fabric includes three distinct layers: a hydrophobic nanofiber layer, a superhydrophilic nanofiber layer, and a conductive layer. As used herein, the term “superhydrophilic” relates to materials that have an excess attraction to water such that there is a complete spreading of water or other polar liquids on the material surface. That is the contact angle of water droplets is approximately zero degrees within 2 seconds. The conductive layer is strategically positioned between the hydrophobic and superhydrophilic layers, creating a sandwich structure of a multilayer composite. This configuration not only facilitates the realization of conductive and electronic sensing functions but also generates a surface energy gradient and a push-pull effect, guiding moisture unidirectionally. The fabric demonstrates remarkable sensitivity within a pressure range of 0 to 2 kPa.

The conductive layer may be coated on one side of either the hydrophobic nanofiber layer or the superhydrophilic nanofiber layer. The fabrication of the hydrophobic and superhydrophilic nanofiber layers employs an electrospinning method, ensuring precision and uniformity. Similarly, the conductive layer is created through electro spraying, contributing to its even distribution and functionality.

The hydrophobic nanofiber layer includes high molecular weight hydrophobic polymers such as polyvinylidene difluoride or its copolymers, polyurethane, or polycaprolactone. On the other hand, the superhydrophilic nanofiber layer consists of high molecular weight hydrophilic polymers, including but not limited to polyacrylonitrile, polyvinyl alcohol, nylon, or polyethylene glycol, and a metal salt.

The conductive coating layer incorporates ultrafine and conductive functional materials like graphene oxide, titanium carbide, carbon nanotube, carbon black, or acetylene black, with a size range of 0.1 to 10 μm. Additionally, a metal salt, specifically lithium chloride or sodium chloride, is integrated into the conductive coating layer to enhance its overall performance.

This biomimetic electronic fabric demonstrates high levels of directional moisture-wicking, offering a combination of sensitivity, rapid response times, and reliable recovery periods when used as a pressure sensor or as an energy harvester/nanogenerator. The materials and fabrication methods employed contribute to a versatile, cost-effective, and scalable solution for various applications, including wearable electronics and smart textiles.

In accordance with a second aspect of the present invention, a method of fabricating the biomimetic, nanofiber-based, and directional moisture-wicking wearable electronic fabric is provided. It incorporates several sequential steps. Initially, either the hydrophobic nanofiber layer or the superhydrophilic nanofiber layer is formed by utilizing the electrospinning technique. Following this, the conductive coating layer is electrosprayed onto a side of either the hydrophobic or the superhydrophilic layer. Subsequently, the remaining layer, whether it be the hydrophobic or superhydrophilic nanofiber layer, is formed on the conductive coating layer, thus establishing the multilayer composite structure with the conductive coating layer positioned as the middle layer. The final step involves the drying of the fabricated layers, resulting in the biomimetic, nanofiber-based, and directional moisture-wicking wearable electronic fabric.

The electrospraying of the conductive coating layer involves a meticulous process. Ultrafine and conductive functional materials, along with a metal salt, are combined in a solution of water and ethanol, augmented by a surfactant to formulate an electrostatic spraying ink. This ink is then electrosprayed onto the side of the hydrophobic nanofiber layer or the superhydrophilic nanofiber layer to create the conductive coating layer.

In particular, the solution of water and ethanol maintains a mass ratio within the range of (5-10):(10-20). A low molecular weight polyvinylpyrrolidone, with an addition amount of 0-5 wt %, serves as the surfactant. The ultrafine and conductive functional material options include graphene oxide, titanium carbide, carbon nanotube, carbon black, or acetylene black, combined with a metal salt, either lithium chloride or sodium chloride. The mass ratio among the ultrafine and conductive functional materials, surfactant, and the solution is maintained at (2-5):(0-1):20.

The electrospraying process is executed by a single-needle electrospraying device, adhering to specific electrostatic spraying conditions. These include a voltage range from 18 kV to 25 kV, an injection pump flow rate ranging from 0.04 mm/min to 0.1 mm/min, and a receiving distance maintained between 5 cm to 15 cm.

For the formation of the hydrophobic nanofiber layer, a first spinning solution is created by adding a hydrophobic polymer and carboxylic carbon tubes to a dimethylformamide and/or acetone solvent, followed by electrospinning. The hydrophobic polymer options encompass polyvinylidene difluoride or its copolymers, polyurethane, or polycaprolactone.

Similarly, the formation of the superhydrophilic nanofiber layer involves preparing a second spinning solution, incorporating a hydrophilic polymer and a surfactant in a dimethylformamide solvent, and electrospinning. The hydrophilic polymer options include polyacrylonitrile, polyvinyl alcohol, nylon, or polyethylene glycol, while the surfactant can be selected from sodium dodecyl sulfonate, sodium dodecyl benzene sulfonate or hexadecyl trimethyl ammonium bromide.

The electrospinning process is characterized by using a single-needle spinning device, maintaining electrospinning conditions comprising a voltage range of 20 kV to 25 kV, a syringe pump flow rate of 0.03 mm/min to 0.06 mm/min, and a receiving distance of 10 cm to 15 cm.

In accordance with a third aspect of the present invention, a biomimetic, nanofiber-based, and directional moisture-wicking electronic skin, in which the aforementioned electronic fabric is integral, is provided. The hydrophobic nanofiber layer is thoughtfully designed to make direct contact with the human skin, enhancing the sensor's functionality and optimizing its performance in various applications.

In accordance with a fourth aspect of the present invention, a single-electrode triboelectric nanogenerator is introduced, featuring the aforementioned biomimetic, nanofiber-based, and directional moisture-wicking wearable electronic fabric and one or more electrodes positioned on the conductive layer. This singular electrode triboelectric nanogenerator is capable of energy harvesting and electronic sensing. Leveraging the fabric's triboelectric properties, this nanogenerator showcases a response time between 20 to 30 milliseconds, a recovery time between 20 to 40 milliseconds and an areal power density within the range of 0 to 21.6 μW m⁻², underscoring its efficiency in converting mechanical energy from moisture movement into a usable power output. This single-electrode configuration streamlines the design and operation, offering a simplified yet powerful solution for diverse energy-harvesting applications.

The nanofiber-based electronic textile of the invention has several advantages: the function of directional moisture wicking, conductivity, sensing physiological signal and harvesting biomechanical energy; the conductive coating layer is prepared by electrospraying, which is simple, controllable; the addition of surfactants contributes to the dispersion of conductive functional materials, spraying uniformity and bonding fastness to the substrate; compared with previous studies, the addition of surfactants can further improve the superhydrophilicity of hydrophilic polymers through intermolecular chemical bonding, without the need for post-treatment.

In the following, the contents of the invention will be further clarified in combination with implementation examples for a better understanding of the invention. However, the content of the invention is not limited to the following embodiments. The technical personnel in this field may make various changes or modifications to the invention, and these equivalent forms are also within the limits of the claims listed in this application.

### EXAMPLES

### Example 1. Fabrication of the biomimetic, nanofiber-based and directional moisture-wicking electronic fabric samples

The fabrication process commences with the dissolution and stirring of 3 g PVDF and 0.1 g carboxylic CNTs in a 16.9 g DMF/Acetone mixed solution (3/2, wt/wt) with the addition of 0.2 wt % LiCl. Using the electrospinning technique, a hydrophobic layer of CNT-modified PVDF (C-PVDF) nanofibers membrane is achieved. Subsequently, an MXene/CNTs conductive ink is electrosprayed onto the C-PVDF nanofibers. For the top layer, 2 g polyacrylonitrile (PAN) is dissolved and stirred in an 18 g DMF solution with 0.2 wt % SDS addition, and a hydrophilic PAN nanofibers membrane is electrospun onto the electrosprayed membrane. During both electrospinning and electrospraying processes, a positive voltage and feeding rate of 25 kV and 0.05 mm min-1, respectively, are set. The tip-to-collector distance is maintained at 15 cm. The electrospraying time for the MXene/CNTs conductive ink varies for different samples (2, 6, and 10 h for sample-1, sample-2, and sample-3, respectively).

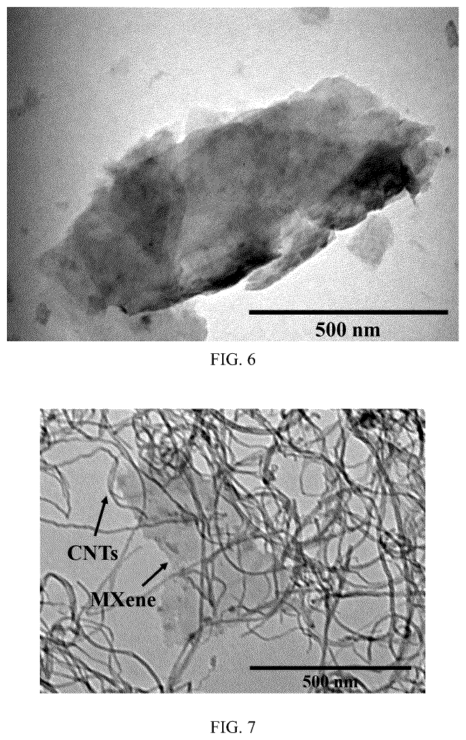

The MXene/CNTs conductive ink is prepared by mixing carboxylated carbon nanotube (CNT) solution and Mxene nanosheet colloidal solution. LiF (1 g) is dissolved in 10 mL HCl (9 M), and Ti₃AlC₂ powders (1 g) are gradually added into the LiF/HCl aqueous solution. The solution is moved into a Teflon autoclave for 24 h at 60° C., followed by sequential washing with 3 M HCl and DI water until pH=7. The obtained black jelly is ultrasonicated in DI water for 2 h under argon protection and centrifuged at 4,000 rpm. The concentration of the colloidal Ti₃C₂Tx nanosheets solution is approximately 8 mg mL⁻¹. The corresponding TEM image of Ti₃C₂Tx nanosheets is shown in FIG. 6. Carboxylated CNTs (30 mg) is dispersed in a 20 mL alcohol/water solution (5/5, v/v), and this solution is added to the Ti₃C₂Tx MXene nanosheets colloidal solution (20 mL). To achieve a stable solution ink, the solution is ultrasonicated for 30 min and stirred for 6 h. The TEM image of the MXenc/CNTs ink are shown in FIG. 7, respectively. The solution conductivity of the MXene/CNTs ink is 9.22 μS cm 1.

In a preferred embodiment, a thin and hydrophobic layer of C-PVDF nanofibers is electrospun onto an aluminum foil as the bottom layer in close proximity to the skin, ensuring a small wetting area and low sweat absorption. Subsequently, the MXene/CNTs conductive ink is electrosprayed onto the C-PVDF nanofibers layer. Electrospraying facilitates control over the spraying time and thickness, preserving the porous nature of the fiber substrate with thin spraying layers. The presence of carboxylic CNTs is instrumental in fostering interactions among C-PVDF, MXenes, and CNTs, preventing the mutual stacking of MXene lamellae and acting as a bridge between MXenes. Finally, a thick and superhydrophilic outer protective layer of PAN nanofibers is constructed by electrospinning PAN precursor solution onto the C-PVDF/MXene-CNTs layer. This electronic fabric effectively pulls sweat away from the skin and sensing layer, enabling quick evaporation with a wider wetting area and ensuring stable bioelectric signal acquisition. The constructed electronic fabric ensures accurate all-over physiological monitoring and achieves biomechanical energy harvesting through the single-electrode triboelectric mechanism. When serving as a pressure sensor, the MXene/CNTs layer functions as the conductive sensing layer, perceiving pressure-induced structural changes and converting them into electrical signals via the interdigital electrode. In its role as a single-electrode triboelectric nanogenerator (STENG), the MXene/CNTs layer acts as the charge acquisition electrode.

### Example 2. Characteristics Evaluation of the Biomimetic, Nanofiber-Based and Directional Moisture-Wicking Electronic Fabric Samples

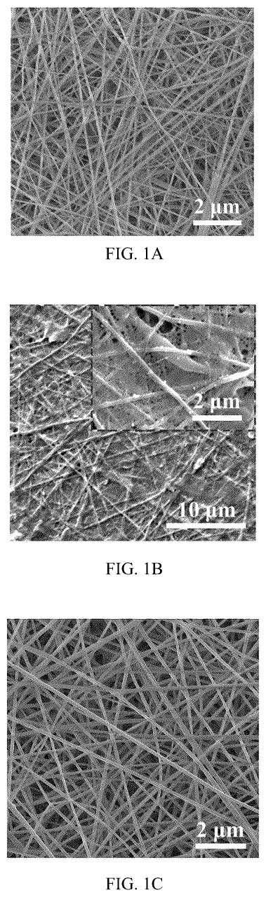

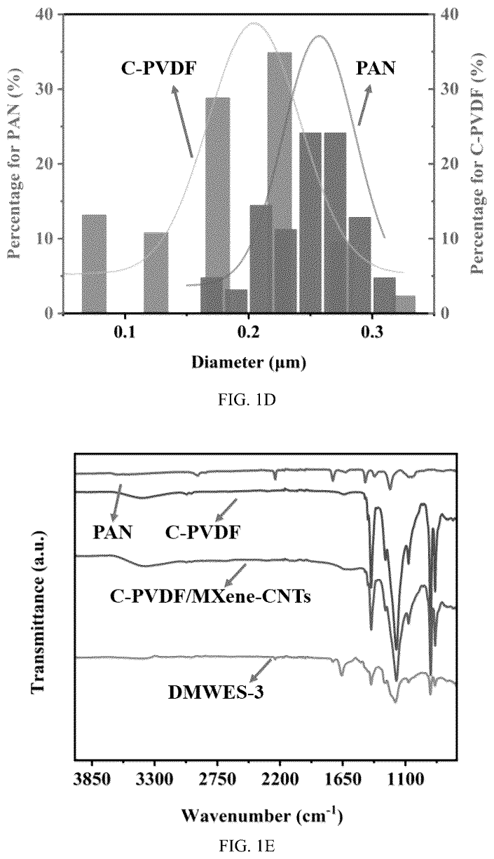

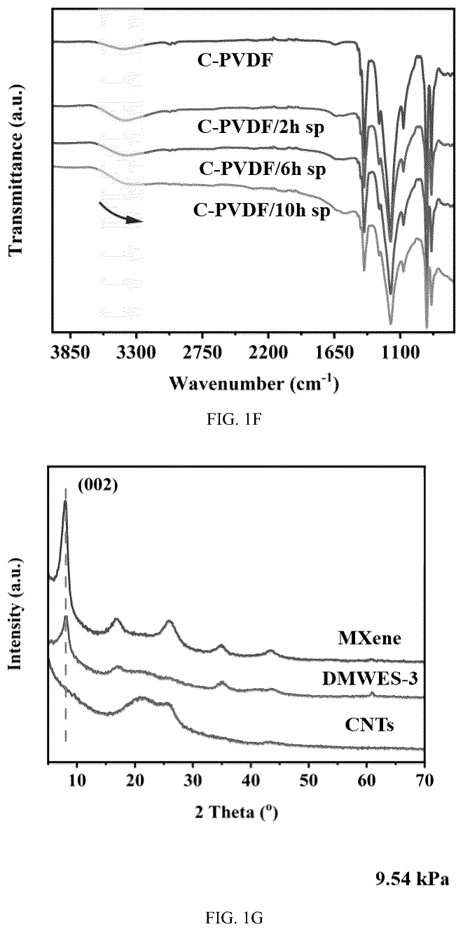

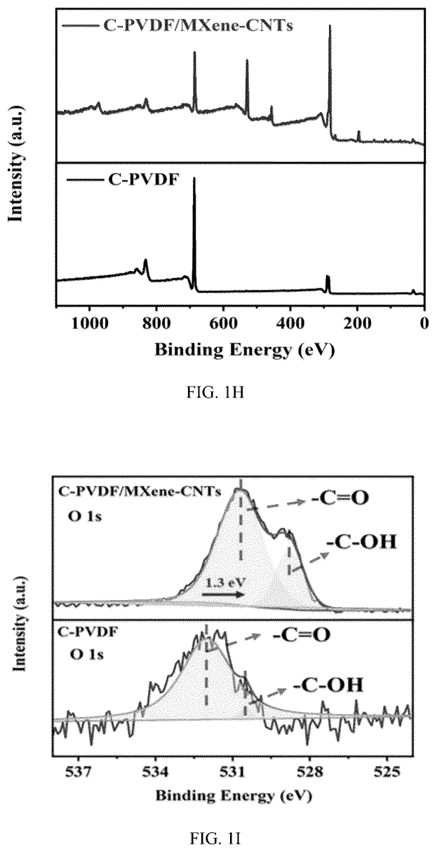

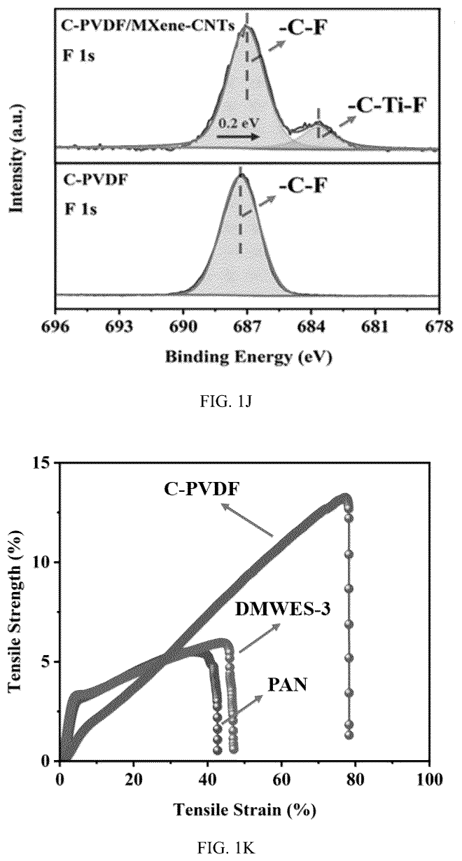

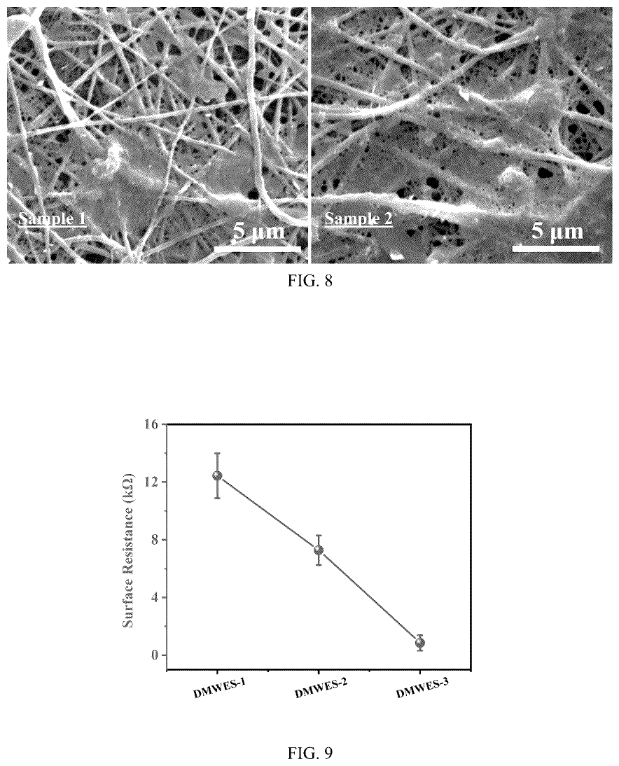

FIG. 1A and FIG. 1C illustrate the morphology of the C-PVDF hydrophobic nanofibers layer and the PAN hydrophilic nanofibers layer, respectively. The morphology is observed utilizing scanning electron microscopy (FEI Quanta 250 e-SEM) and transmission electron microscopy (TEM, Philips CM20). The nanofibers exhibit a randomly distributed nonwoven fabric morphology. In FIG. 1B, the morphology of electrosprayed MXene/CNTs ink (sample-3) on the C-PVDF substrate is depicted; the MXene nanosheets and CNTs are uniformly distributed on the hydrophobic layer, maintaining the porous structure of hydrophobic layers simultaneously. The SEM images of sample-1 and sample-2 with different electrospraying times are shown in FIG. 8. The most uniform deposited MXene/CNTs are obtained by sample-3 with an increased electrospraying time.

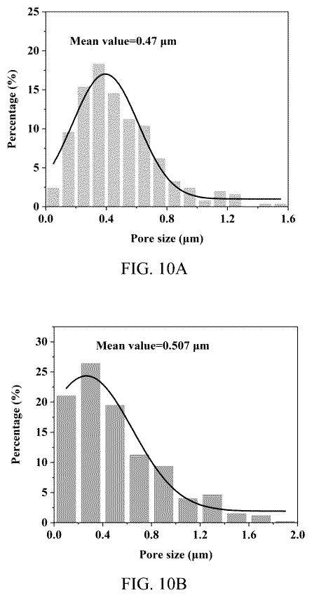

Additionally, the surface resistance changes of the DMWES are shown in FIG. 9. The surface resistance of the nonwoven membrane is measured by the digital four-probe tester. The larger surface resistance of sample-1 and sample-2 should be ascribed to the incomplete conductive paths with low MXene/CNTs electrospraying time. The fiber's average diameters for the C-PVDF layer and the PAN layer are about 200 and 260 nm, respectively. The C-PVDF layer changes by approximately an order of magnitude, while the PAN layer shows more uniformly sized fibers (FIG. 1D). Meanwhile, as shown in FIG. 10A and FIG. 10B, the mean pore diameters generated in the PAN layer and C-PVDF layer due to fiber interlaced with each other are 0.47 and 0.507 μm, which grow by layer and may be conducive to the enhanced capillary force of the hydrophilic pores.

As shown in FIG. 1E, the chemical properties of the fabrics are analyzed using a PerkinElmer FT-IR Spectrometer. The spectrum shows the characteristic stretching vibration of the nitrile group (C═N) of PAN at 2,240 cm 1. The spectrum of C-PVDF nanofibers displays the typical in-plane bending of the —CH₂ group at 1,405 cm 1 and symmetric stretching of the —CF₂ group at 1,170 cm 1. A wider peak shifting to a lower wavenumber at 3,450 cm⁻¹ of —OH group indicates the possible existence of hydrogen bonds between the C-PVDF nanofibers matrix and MXene/CNTs inks (FIG. 1F). FIG. 1G shows the X-ray diffraction (XRD) patterns, performed by a D2 Phaser XE-T X-ray diffractometer system with a scan rate and range of 20 set as 5° min 1 and 5°-70°, of MXene, CNTs, and sample-3; the characteristic peak of Ti₃C₂Tx at 20 of 6.9° indicates the diffraction from the (002) phase of Ti₃C₂Tx.

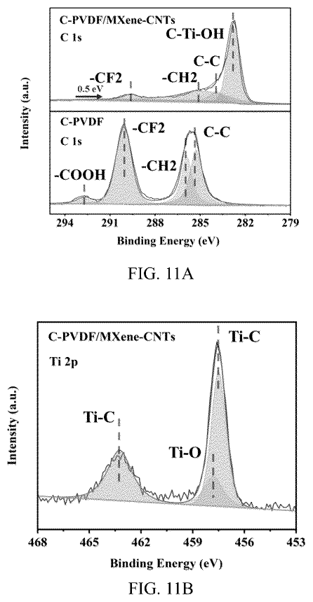

The X-ray photoelectron spectroscopy (XPS) survey scan exhibits the coexistence of C, O, F, and Ti elements in C-PVDF/MXene-CNTs (FIG. 1H). FIGS. 1I and 1J exhibit the high-resolution O 1s and F 1s spectrum of C-PVDF and C-PVDF/MXene-CNTs. For O Is spectrum, the peaks at 532 and 530.5 eV are associated with —C═O bond and —C—OH bond of C-PVDF (FIG. 1I), respectively. The peaks are negatively moved to lower binding energy by about 1.3 cV in C-PVDF/MXene-CNTs via the —F . . . H—O interaction between the —Fon C-PVDF and —OH group of carboxylated CNTs. The —C—F peak of C-PVDF from F Is spectrum is shifted to a lower binding energy by ˜0.2 eV in the electrosprayed sample (FIG. 1J). The consistent result is also observed from the high-resolution C Is spectrum (FIGS. 11A-11B). These results demonstrate the formation of a stable interface interaction between C-PVDF and MXenes/CNTs.

The tensile strength of the nanofiber membrane is of great significance to the wearability evaluation of the fabric-based wearable electronics. It can be observed from FIG. 1K that the strength of the DMWES membrane is slightly enhanced to 6 MPa because of the high strength of the hydrophobic C-PVDF layer. Good water vapor transfer rate (WVTR) has a significant impact on the wearing experience. The fabric with porous gradient structures and asymmetric wettability can continuously transport water vapor from the hydrophobic C-PVDF layer to the hydrophilic fibrous PAN layer rapidly. As a result, the WVT of sample-3 at 25° C. and 50% relative humidity (RH) can attain a WVTR of 13.99 kg m⁻² d⁻¹, which decreases slightly with the increase in the electrospraying MXene/CNTs amount (FIG. 1L). The value is comparable to the WVTR of commercial e-PTFE (15.1 kg m⁻² d⁻¹), indicating good wear comfort.

### Example 3. Directional moisture-wicking ability of the biomimetic, nanofiber-based and directional moisture-wicking electronic fabric

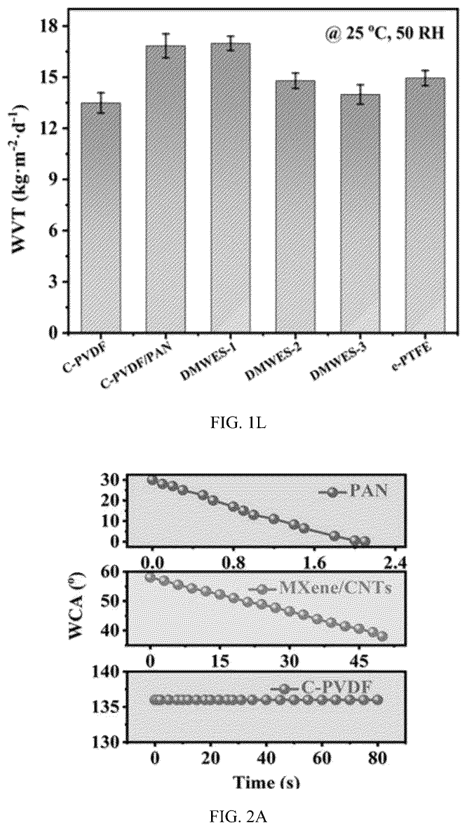

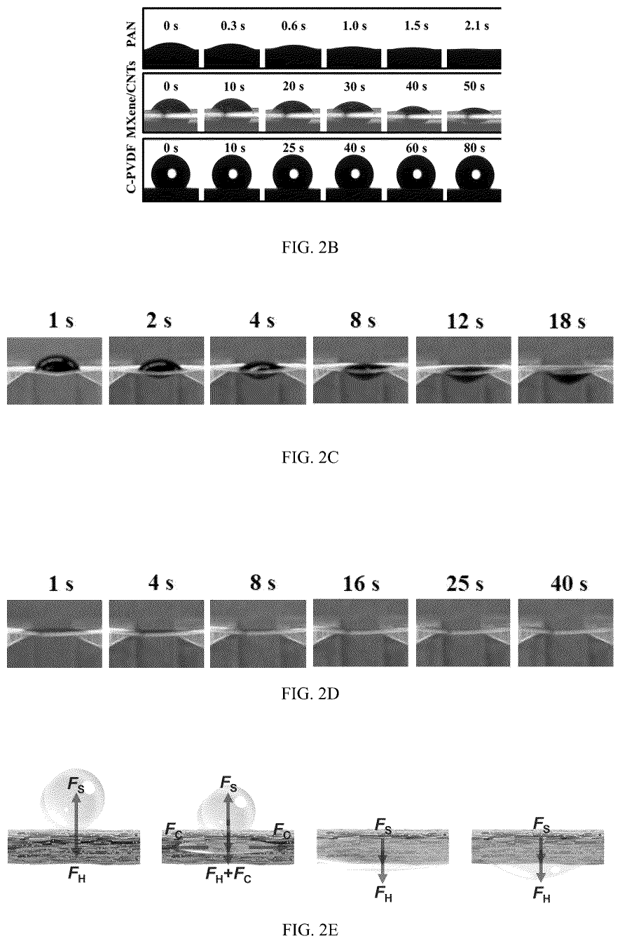

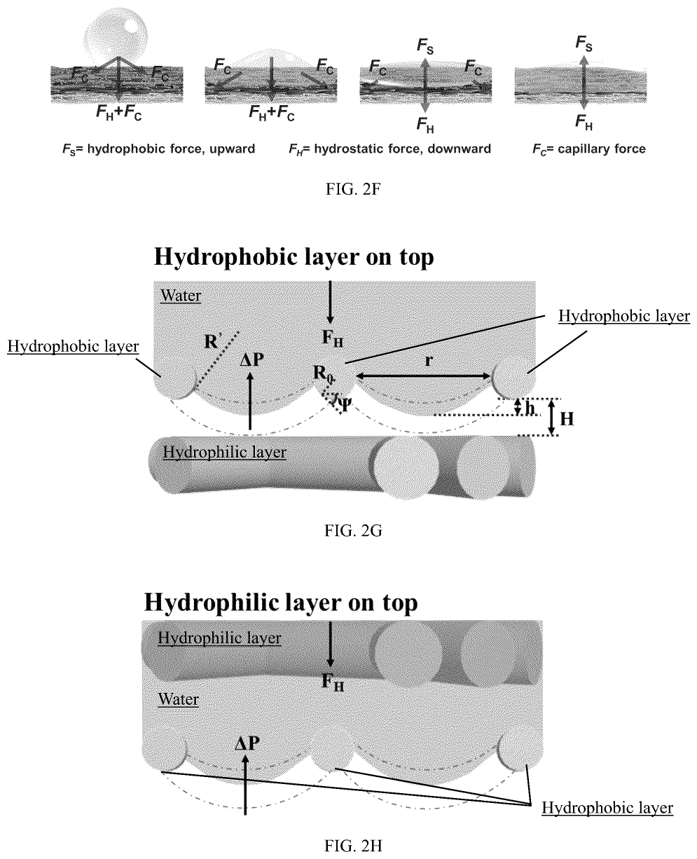

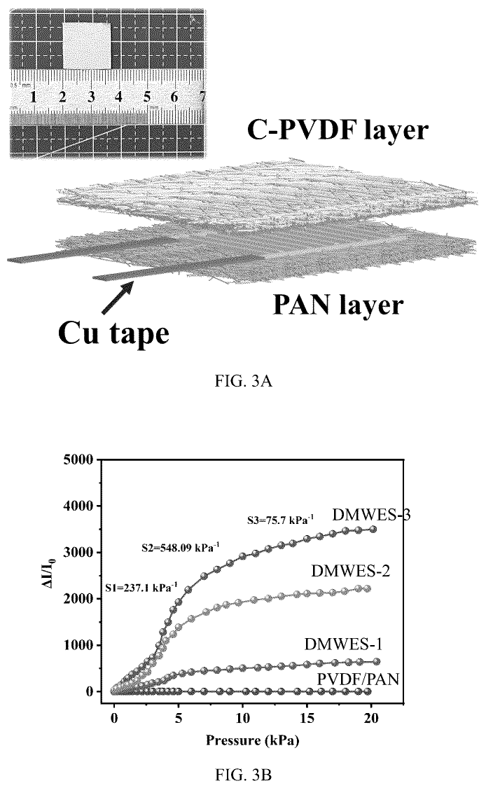

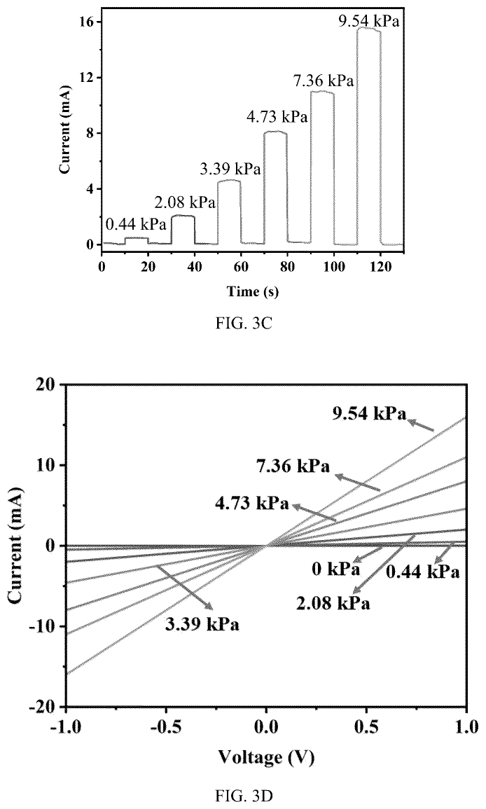

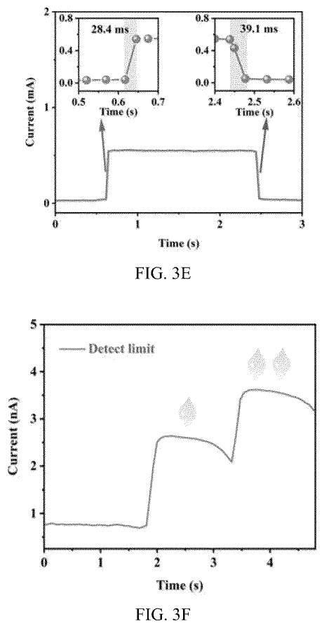

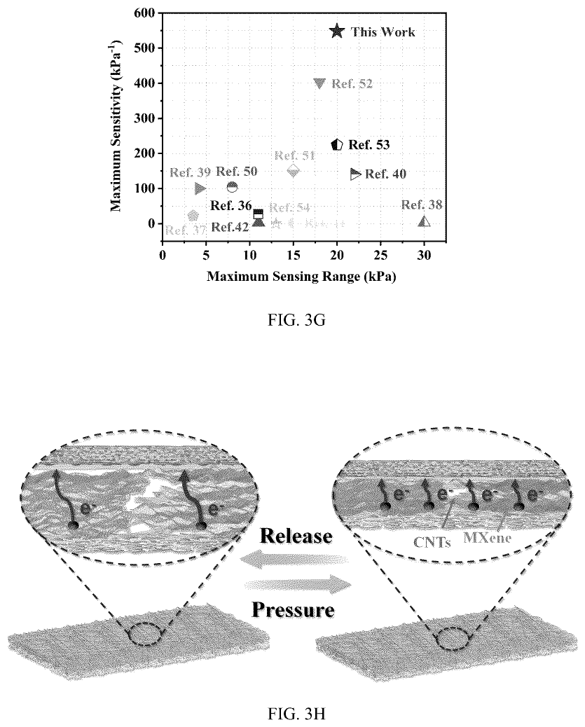

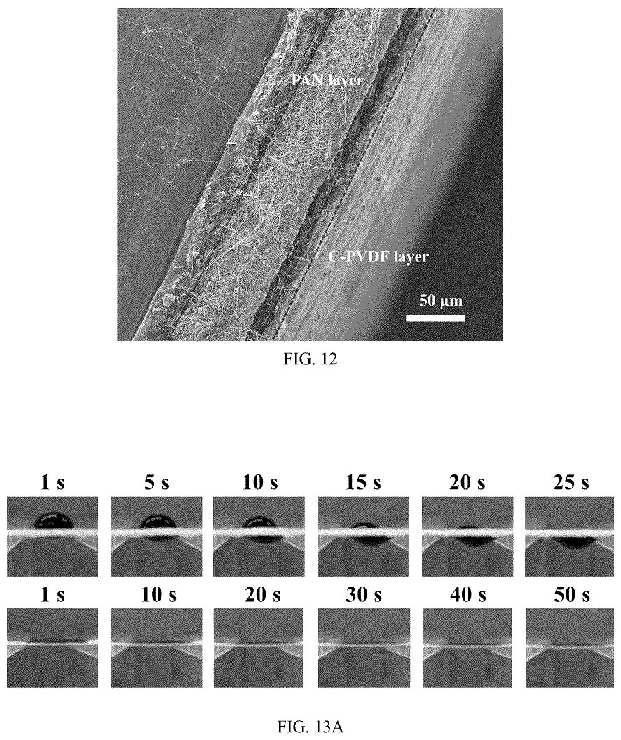

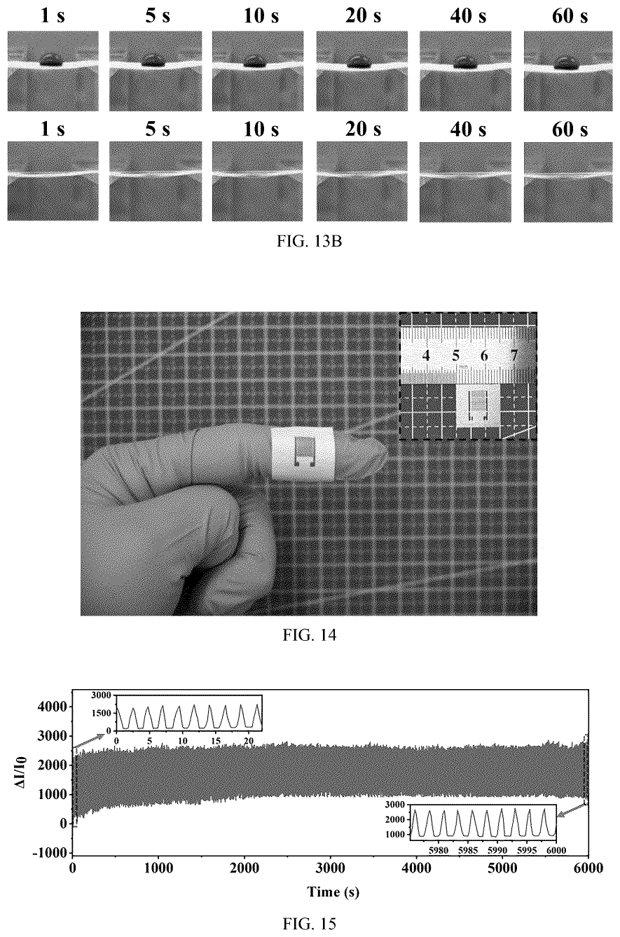

In order to evaluate the directional water transport performance in the electronic fabric, the wettability of the hydrophilic and hydrophobic nanofiber membranes is evaluated first. FIG. 12 exhibits the cross-sectional image of the electronic fabric with the thickness of about 15 and 60 μm for the hydrophobic C-PVDF nanofbers layer with MXene/CNTs conductive layer and superhydrophilic PAN nanofbers layer, respectively. FIGS. 2A-2B show the water contact angle (WCA) change of each single layer with time increasing. On the PAN nanofibrous layer, the WCA dynamically decreases from ˜31° initially to 0° within 2.1 seconds, showing the superhydrophilicity of the nanofibrous PAN. The C-PVDF layer indicates the stable hydrophobic behavior. The water droplet with an initial WCA of 138° and no obvious spreading on the hydrophobic C-PVDF nanofibers are observed. The water transport capability of the electronic fabric is monitored by the dynamic transfer process of water droplet on both sides of fabric. When a water drop contacted the hydrophobic C-PVDF layer, the water is pumped over the hydrophobic side and wetted the superhydrophilic PAN nanofbers side in about 18 s (FIG. 2C). In contrast, when the electronic fabric is flipped and the water droplet contacted the superhydrophilic nanofibrous PAN layer, the water droplet is blocked and spread over the superhydrophilic nanofibers without penetrating the hydrophobic C-PVDF nanofibers (FIG. 3D). These results suggest that the electronic fabric can transport liquid unidirectionally from the hydrophobic side to the superhydrophilic side. Therefore, the thickness of the C-PVDF nanofibers layer can be regulated to assess the thickness effect on directional water transport performance. When the thickness of the C-PVDF increased to 18 μm, the water droplet transferred from the upper hydrophobic layer to the downward hydrophilic layer in 25 s because of great hydrophobic force (FIG. 13A). When the thickness of the C-PVDF increased to 30 μm, the water droplet cannot be transferred from the upper layer to the downward layer (FIG. 13B). In the reverse direction, the water droplet cannot penetrate into the downward hydrophobic nanofibers layer in these two conditions, and it can just spread on the hydrophilic nanofibers layer.

There is a possible mechanism for both conditions from the macroscopic force analysis in FIGS. 2E-2F. When the hydrophobic C-PVDF nanofibers layer is facing upward and the droplet touches the surface, the droplet will be subjected to the upward hydrophobic force (FS) and the downward hydrostatic pressure (FH). The FH is directly proportional to the height and mass of the droplet. As the droplet increases in size and time, the FH will break through the water transfer barrier force because of the FS of the C-PVDF nanofibers. The water will be trapped by the hydrophobic pores and come to contact the underlying superhydrophilic nanofibers. After that, the droplet will be exposed to strong capillary force (Fc, see the below Eq. 1) in all directions except upward, which is generated by the Laplace pressure.

$$Fc = \frac{2 γcos θ}{r} \qquad ( 1 )$$

where r, γ, and θ are the pore radius, the liquid surface tension, and the liquid contact angle, respectively.

Obviously, Fc is inversely proportional to the pore size, demonstrating that the gradient porous structure plays an important role in the water transport process. The decreased pore size of the PAN nanofibers resulted in the increase in the Fc in both the vertical and the horizontal directions. The Fc will continually pull water into the internal superhydrophilic nanofibers layer through numerous pores of the C-PVDF nanofibers. And then the Fₛ will reverse and further facilitate the downward transfer of water droplets. Finally, the liquid will drip from the superhydrophilic PAN nanofibers (FIG. 2E). When the DMWES is reversed, the droplet will be subjected to the strong capillary force Fc of the PAN nanofibers from every direction except upward until the superhydrophilic nanofibers are fully wetted. The lower hydrophobic C-PVDF nanofibers substrate will then exert an upward Fₛ on the contacted liquid to prevent the liquid from dripping (FIG. 2F).

The model of the superhydrophilic and hydrophobic nanofibers are further simplified in microscopic scale to understand the key parameters of directional water transport. As shown in FIG. 2G, when the hydrophobic nanofibers face up, the liquid-solid-gas three-phase contact line (TCL) will go lower toward an equilibrium position after dipping water and form the convex surface between the hydrophobic fibers as the initiating surface. The convex surface is then subjected to an upward force (ΔP) calculated by the following Eq. 2

$$Δ P = \frac{γ}{{R}^{′}} = \frac{2 γ sin [ θ ( ψ ) - ψ ]}{r + 2 {R}_{0} ( 1 - sin ψ )} \qquad ( 2 )$$

where r is the pore diameter between the hydrophobic fibers, v is the local geometrical angle, and Ro is the fiber radius. ΔP corresponds to the value of FH when TCL reaches an equilibrium state.

The TCL goes lower with the increase in F_{H}, and the vertical distance h between the bottom of the hydrophobic nanofibers and the convex apex increases. The distance between hydrophobic and superhydrophilic nanofibers is H. To achieve directional water transfer, h=H must be satisfied so that the liquid can reach the superhydrophilic fibers. Then, the reverse hydrophobic force (Fₛ) and strong Fc of hydrophilic layer can induce the push-pull effect to drag the liquid to the pores of the hydrophilic nanofibers. Nevertheless, when the thickness of hydrophobic nanofibers increases, ΔP will increase because of the decreased r value by mutual stacking of nanofibers; hence, h will decrease accordingly. The h will be much smaller than H in this case, so directional water transport cannot be implemented. When the hydrophilic layer is on top (FIG. 2H), water droplets will not drip down from under the hydrophobic fibers, because the air can be regarded as absolutely superhydrophobic and h is infinitely less than H. Herein, the directional moisture-wicking multilayered nanofibers membrane can meet the following four requirements:
(1) The hydrophobic nanofibers are hydrophobic enough to provide adequate Fₛ;
(2) The hydrophobic fibers have to be thin to allow water to go through quickly;
(3) The hydrophobic layer has numerous pores, and the gradient pore size from the hydrophobic layer to the hydrophilic layer will induce higher capillary pressure as the driving force to extract the water efficiently;
(4) The hydrophilic layer is relatively thick and has excellent hydrophilicity to directly extract the moisture.

### Example 4. Pressure sensing performance of the biomimetic, nanofiber-based and directional moisture-wicking electronic fabric

To test the sensing performance of the electronic fabric, the piezoresistive effect and the triboelectric effect of the fabricated electronic skin are cooperatively utilized to realize all-range healthcare monitoring, and biomechanical energy harvesting. For the pressure sensing tests, conductive Cu tapes are adhered onto two electrodes of the interdigital electrode of electronic fabric, and the electronic fabric is encapsulated by the medical grade adhesive tape on the top side to ensure to ft skin. Firstly, the electromechanical properties of electronic skin are measured by monitoring the relative resistance variation or sensitivity (ΔI/Iₒ, ΔI=I-Iₒ, Iₒ indicates the original current, I represents the current under pressure). FIG. 3A shows the schematic diagram of the sensing elements, and the inset is the picture of real product with the size of 1.6×1.6 cm². An interdigital Au electrode is sputtered on the PAN nanofibrous membrane to contact the MXene/CNTs sensing layer and collect the electric signal (FIG. 14). FIG. 3B shows the sensitivity curve of the electronic skin with different MXene/CNT contents under wide range of pressure. The sample-3 is observed to have the highest sensitivity value in comparison with the others, which can be ascribed to the uniformly distributed MXene/CNTs sensing layer. The sample-1 and sample-2 show lower sensitivity in comparison with sample-3, which can be attributed to the uneven spraying of MXene/CNTs on the C-PVDF nanofibers matrix and thus, larger interface resistance and poor ohmic contact between the conductive sensing layer and interdigital electrode, resulting in low sensitivity. The sensitivity curve can be split into three parts: S1 exists in the low-pressure area (0-3.20 kPa), S2 lies in the middle-pressure area (3.20-6.30 kPa), and S3 lies in the high-pressure area (6.30-20.0 kPa). The corresponding sensitivity of S1, S2, and S3 is 237.1, 548.09, and 75.7 kPa⁻¹, respectively. In the low-pressure range, the current response and sensitivity of the electronic skin are relatively lower than the sensitivity at the medium-pressure range, which is due to the small air gap between the interdigital electrode and the conductive MXene/CNTs layer and the energy absorption effect of the nanofibers. The following measurements of other performance are herein determined by the sample-3. The current-time (I-T) curves show a gradual increasing trend in the current under the correspondingly increased external pressure (FIG. 3C). FIG. 3D exhibits the current-voltage (I-V) curves of the sample-3 with the voltage sweeping from −1.0 to 1.0 V, showing the proportional increasing dependence of the voltage by the current under different pressure. This finding demonstrates a good ohmic contact between the conductive sensing layer and the interdigital Au electrode, enabling the electronic fabric to distinguish different pressures over a wide range. Furthermore, the electronic skin displays a fast response (28.4 millisecond) and recovery time (39.1 millisecond) because of the superior contact resilience of the C-PVDF nanofibers film and interdigital electrode (FIG. 3E). The lowest detection limit of the electronic fabric is one of the critical factors in determining the scope of electronic skin applications.

Furthermore, two drops of water are utilized to evaluate the detection capability of the electronic fabric at extremely low pressures. As shown in FIG. 3F, the slight force imposed by a drop of water (˜5 Pa) can be exactly detected by the electronic fabric. By comparing the maximum sensing range and sensitivity with the previous studies, the electronic fabric of the present invention exhibits comprehensive advantages (FIG. 3G), showing great potential in various application scenarios.

The long-term piezoresistive stability of the electronic fabric is further evaluated by a continuous compression and separation test at 10 kPa; the current response under pressure is stable even after 3,000 cycles (FIG. 15). For a more comprehensive performance evaluation, various parameters, including response/recovery time and detection limit, are systematically compared and presented in Table 1. The results depicted in Table 1 clearly demonstrate that the C-PVDF/MXene-CNTs of the present invention exhibits superior performance across all parameters in comparison to other Ti₃C₂Tx or MXene materials encapsulated with PET, PE, or PDMS.

TABLE 1
Sensing performance evaluation

| | | Detect | Maximum | Maximum | Response/ |
|---|---|---|---|---|---|
| | | limit | sensitivity | sensing | recovery |
| Materials | Encapsulation | (Pa) | (kPa⁻¹) | range (kPa) | time (ms) |
| C-PVDF/MXene- | Nonwoven | 5 | 548.09 | 20 | 28.4/39.1 |
| CNTs/PAN | Nanofibers | | | | |
| PAN/Ti₃C₂Tₓ | PET | 1.5 | 104 | 8 | 30/20 |
| MXene/PDMS | PE | 4.4 | 151.4 | 15 | 125/104 |
| MXene/PVA/PDMS | PDMS | 0.88 | 403.46 | 18 | 105.3/99.3 |
| CNT/Ti₃C₂Tₓ | PDMS | — | 0.245 | 13 | —/— |

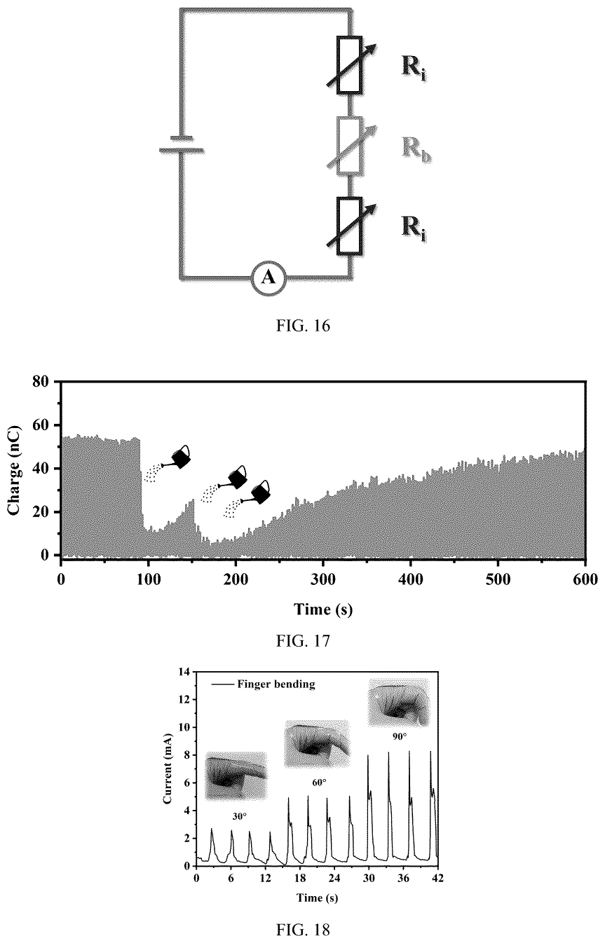

The proposed sensing mechanism of the electronic fabric is shown in FIG. 3H. The electronic fabric is equivalent to a serial circuit that contains three variable resistances. The equivalent circuit of the resistance is exhibited in FIG. 16. When the external pressure is imposed on the electronic skin, the contact point in the conductive network of C-PVDF/MXene-CNTs increases relatively. A significant decrease in the contact resistance (Rᵢ) and bulk resistance (R_{b}) of the electronic fabric will be observed. The Rᵢ change of the electronic fabric is determined by the contact dimension between the MXene-CNTs electrospraying side and interdigital electrode. The sensing layer and the electrode are not in close contact with each other in the original no-pressure condition; hence, a high Rᵢ exists initially at their interface, and then, it decreases under increased pressure. Rᵢ exhibits a significant response to tiny pressure. When the electronic fabric is exposed to higher pressure, the Rᵢ variation will attain saturation rapidly, and R_{b} will decrease gradually under higher pressure, giving the electronic fabric a wider detection range.

### Example 5. Triboelectric Nanogenerator Performance of the Biomimetic, Nanofiber-Based and Directional Moisture-Wicking Electronic Fabric

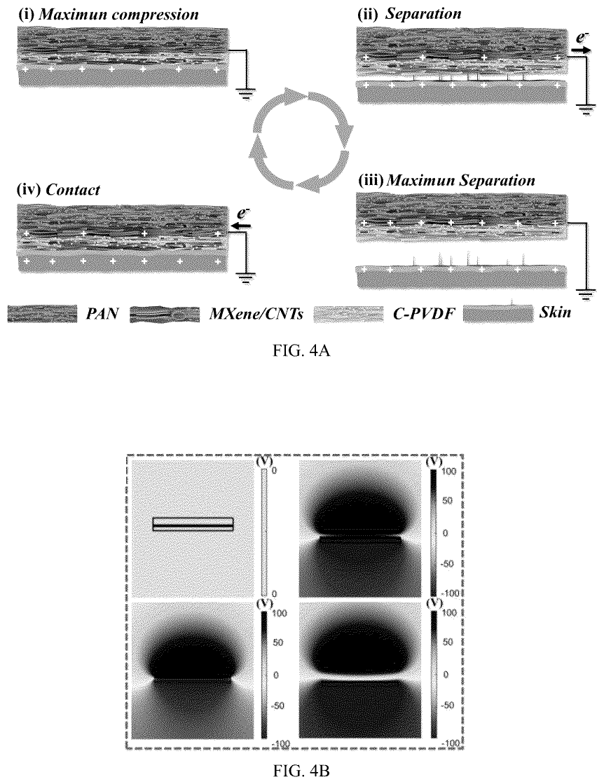

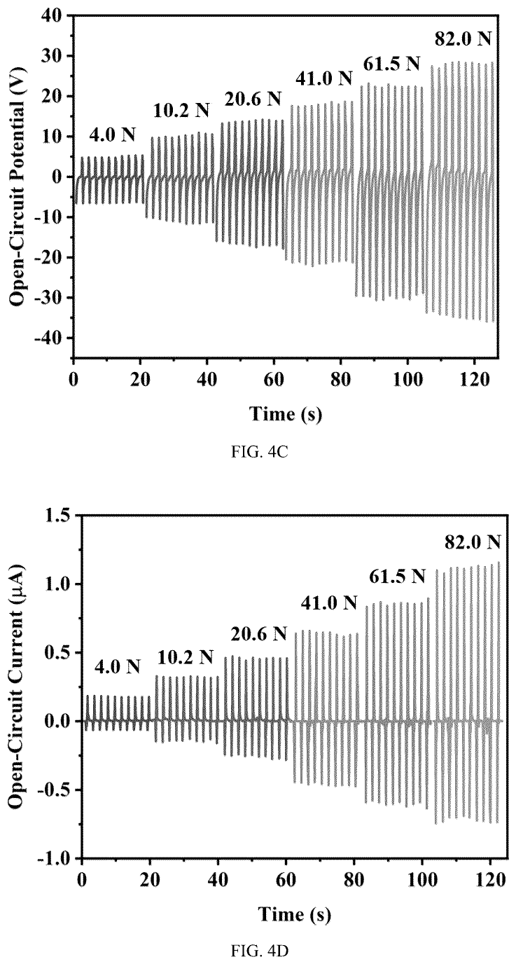

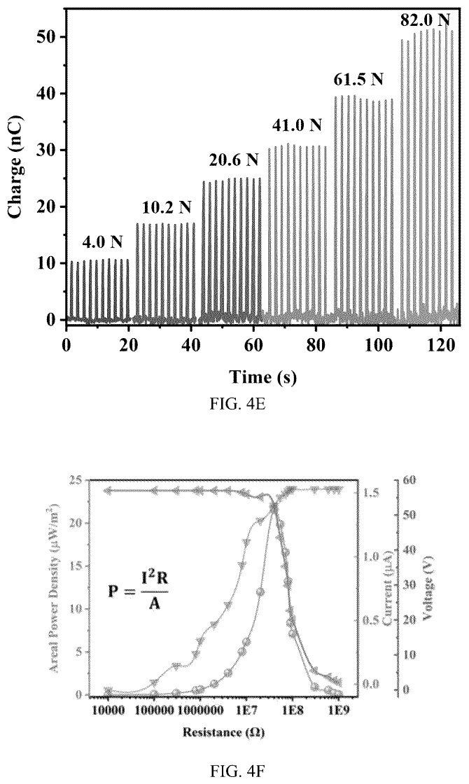

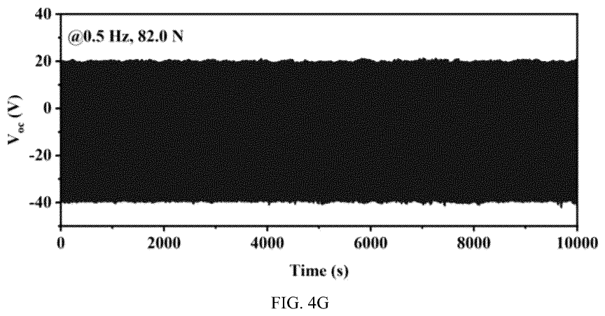

On account of the high conductivity of MXene/CNTs electrospraying layer and strong electronegativity of the MXene/CNTs, C-PVDF, and PAN layers, the electronic fabric is further explored based on the single-electrode triboelectric mechanism for biomechanical energy harvesting, in which the MXene/CNTs electrospraying side acted as the electrode. FIG. 4A demonstrates that the electronic fabric in single-electrode pattern includes two sections: MXene/CNTs electrospraying side as electrode layer, the electronic fabric and human as negative triboelectric and positive layers, respectively. When the skin (or other counterparts) is in contact with the electronic fabric, the skin will transfer the charges to the electronic fabric due to the high surface electron affinity of the electronic fabric. Once the separation occurs, the potential difference is generated. Hence, the electronic fabric continuously produces an alternate electricity (AC) signal through the entire cycle of contact and separation processes. FIG. 4B presents the simulated electric potential distribution by COMSOL multiphysics, explaining the electricity generation process of during the contact and separation process of triboelectrification quantitatively. With the maximum degree of contact and separation, the simulation output of single-electrode triboelectric nanogenerator (STENG) reaches the maximum value of about 200 V. FIGS. 4C-4E demonstrate the triboelectric output of the electronic fabric when pairing with aluminum foil as the positive counterpart, the output performance shows the linearly proportional force-dependent relationship until 82.0 N, which can generate the maximum output including the V_{oc}=62 V, I_{sc}=1.6 μA, and Q_{sc}=49 ηC at the force of 82.0 N, respectively. Water spraying treatment is also conducted for the triboelectric nanogenerator (TENG to simulate the influence of water and sweating on the output performance. When spraying water on the TENG during the test, the TENG output is decreased rapidly and then, gradually recovered after a certain period of self-drying, which can be restored to about 80% of the original state in 10 min (FIG. 17). FIG. 4F shows the output voltage, current and areal power density of the electronic fabric with external resistance loadings from 10 kΩ to 1 GΩ. The areal peak power density of the electronic fabric is calculated from the equation of P=PR/A, where A, R, I, and P represent the contact area, resistance loading, output current, and power density of the electronic fabric, respectively. The areal peak power density of the electronic fabric can reach a peak value of 21.6 μW m⁻² at the external resistance loading of 40 MΩ. Furthermore, the durability of the electronic fabric is demonstrated in FIG. 4G. The generated Vₒₓ: nearly appears as a constant of ˜60.5 V after 5,000 continuous cycles at a frequency of 0.5 Hz and 82.0 N loading force, showing excellent mechanical robustness and stability.

### Example 6. All-range healthcare sensing of the biomimetic, nanofiber-based and directional moisture-wicking electronic fabric

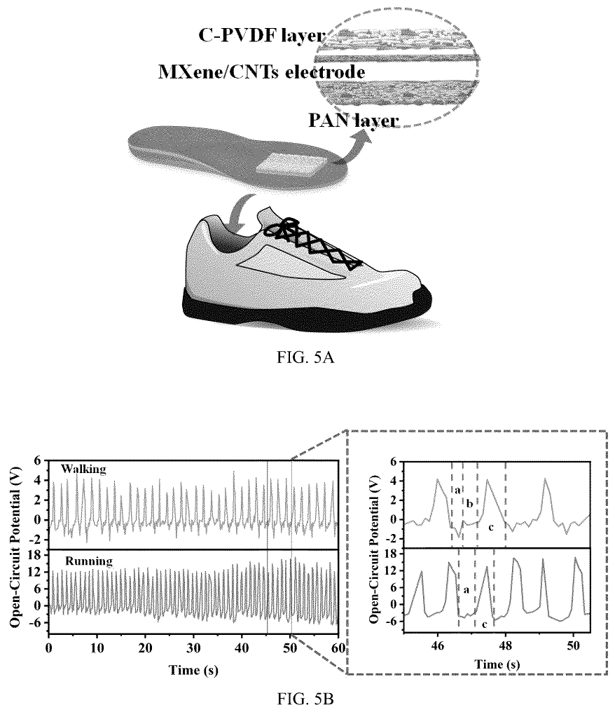

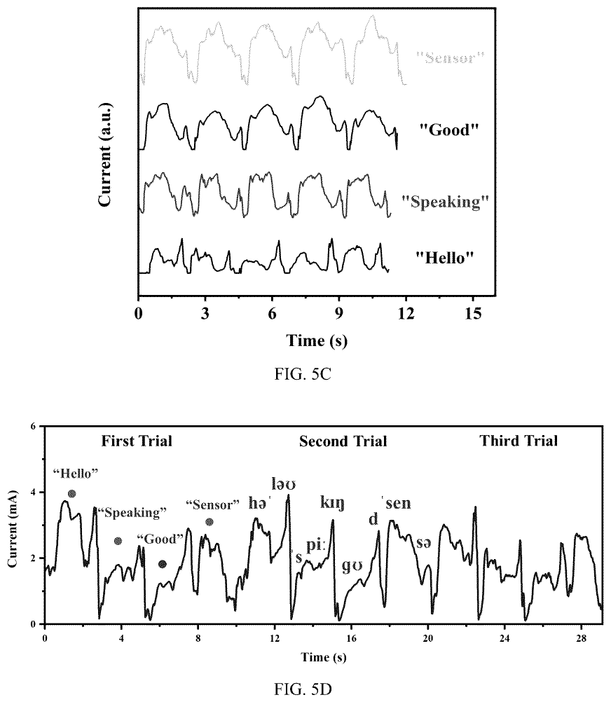

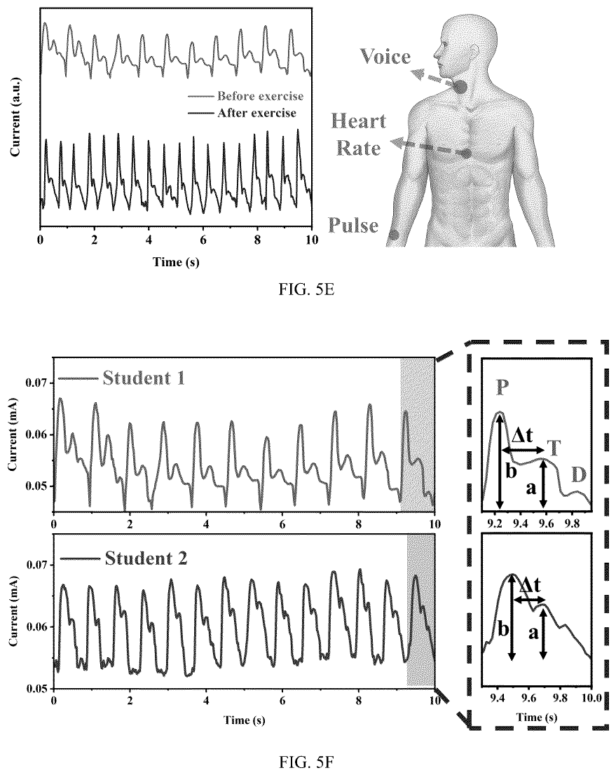

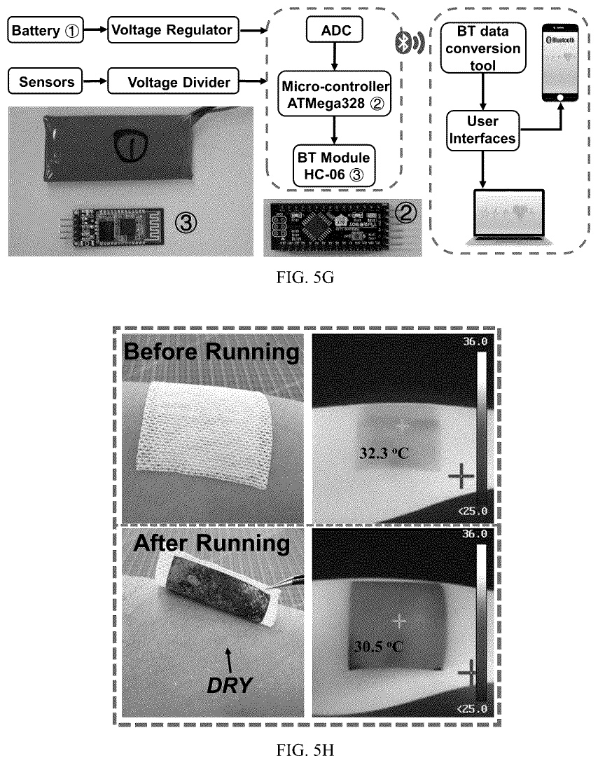

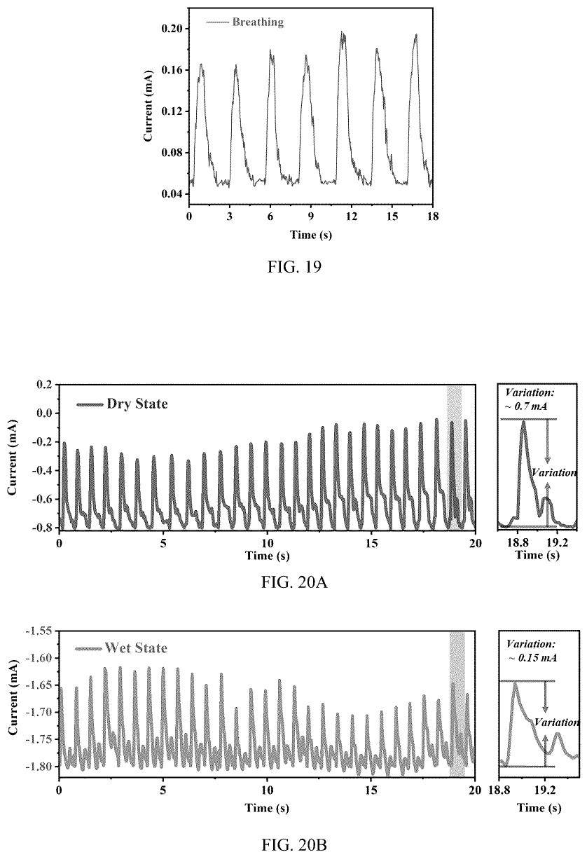

Based on the superior pressure sensing and triboelectric performance of the electronic fabric, the electronic fabric can realize the all-over healthcare sensing with these two mechanisms. In FIG. 5A, the electronic fabric is used to gather gait information and biomechanical energy based on the single-electrode triboelectric sensing mechanism during running exercise, which is simply pasted onto the internal sole of the running shoe. Distinct signal patterns generated when the volunteer is walking at different paces, which can be acquired by analyzing the frequency and amplitude of the obtained signals easily (FIG. 5B). The V_{oc} signal of running (1 Hz frequency) is approximately two times higher than the generated signal of walking (0.5 Hz frequency). Additionally, the walking pace can be resolved into three sections from the one-step signal in FIG. 5B (i.e., a. stomping, b. intermission, c. lifting and striding). Two inverse V_{oc} peaks can be observed in stage a and stage c, indicating the imposed pressure by feet during walking. The intermission can be applied to demonstrate the walking pace. The stage b disappears when the feet frequency reached 1 Hz, indicating fast running. The walking information is of great value to physicians and coaches in the physical therapy of patients with lower extremity disorders and scheduling training sessions for athletes. In addition, the electronic fabric can achieve the accurate physiological monitoring of weak bio-signal based on the piezoresistive pressure sensing. The electronic fabric exhibits clear and steady signals of various degree of joint motions (FIG. 18) and displays a high sensitivity for articular nimbleness monitoring. FIG. 19 shows the 7 cycles of breathing signal in 18 s, which is consistent with the normal adult breathing frequency of 16-24 cycles in one minute. FIG. 5C shows the electronic fabric attached onto the volunteer's throat to monitor the tiny movement of vocalization. The electronic fabric produces apparent signal patterns for different word vocalization, i.e., “hello” “speaking” “good,” and “sensor,” demonstrating the potential application in AI and voice recognition. FIG. 5D shows the signal record of words spoken together in three trials, indicating the consistency of the voice signal. Interestingly, the experimental signal results represent the similar characteristic peaks existing in corresponding tones of word signals. Pulse waves provide vital information on heart conditions and related diseases. Generally, a radial artery wave can be observed with the characteristic percussion, tidal, and diastolic peaks (P-wave, T-wave, and D-wave, respectively). Further, the electronic fabric is applied to monitor human pulse waves by attaching the electronic fabric to the wrist (FIGS. 5E-5F). FIG. 5E shows the pulse wave of a 28-year-old male man before and after work-out. The pulse rate and amplitude are both increased after a 10 min rope skipping exercise, and the waveform is changed as well. Specifically, the pulse rate increases from 66 beats per minute (BPM) in the resting state to 104 BPM in the exercising state. The tidal peak comes to be blurred and the relative strength of the diastolic peak to the percussion peak reduced on the waveform. It is worth noting that the all-fiber based electronic fabric exhibits stable pulse signal even after exercise, demonstrating that the directional moisture transport capability enables the relatively stable test environment of MXene/CNTs sensing layer. The pulse monitoring performance of the DMWES in the simulated sweat environment with NaCl solution is also evaluated. As shown in FIGS. 20A-20B, it is observed that the electronic fabric can still clearly detect pulse signals in the simulated sweat (wet state), comparing to dry state. But the current intensity is higher because of more conductive paths as water penetrated into the conductive and hydrophilic layers. The increased internal contact interfaces result in the decrease in the pulse peak intensity variation. In FIG. 5F, the pulse wave of two users (a 28-year-old male man and a 30-year-old male man for user 1 and user 2, respectively) is tested. The analysis of stationary signals shows radial artery frequencies of ˜66 and ˜84 BPM, respectively, indicating the good health state of two users. As presented in the enlarged pulse of FIG. 5F, the typical peaks (P-wave, T-wave, and D-wave) are clearly captured and distinguished. The significant health indicators like refection index (R.I., Eq. 3) and stiffness index (S.I., Eq. 4) are also observed from the response time by resolving the two distinct peaks of P-wave and T-wave (Δt), and the intensity of P-wave and T-wave (b and a, respectively). The stiffness of the vessels is assessed by the measurement of R.I. and S.I.:

$$R . I . = \frac{a}{b} \times 100 \% \qquad ( 3 )$$

$$S . I . = \frac{Subject height}{Δ t} ( m / s ) \qquad ( 4 )$$

The R.I. and S.I. values are computed to be 50.1% and 5.40, 64.3% and 9.38 for user 1 and user 2, respectively. The results indicate the healthy status of the two users. The higher the R.I. and S.I. values, the higher the risk of cardiovascular disease like hypertension, cardiovascular infarction.

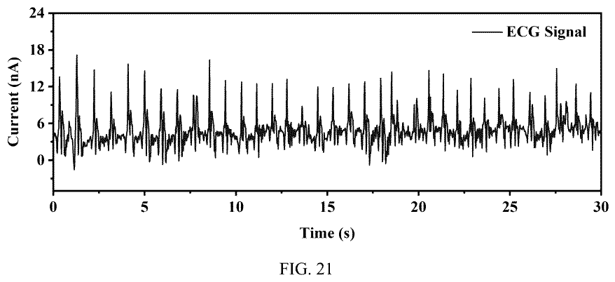

A wearable physiological monitoring system connecting the electronic fabric is further developed for ECG detection. As shown in FIG. 5G, the main components of the system are exhibited, including data acquisition and processing unit, wireless data transmission module, etc. A circuit that used a frequency divider circuit structure to transform the resistance of the electronic fabric into a voltage, which is then converted to the digital domain by an analog-to-digital converter (ADC), is also designed. The analog output of the driver circuit is synchronously digitized using a high-precision ADC chip. The digital signals are processed by a microcontroller (ATMega328) and then, transmitted wirelessly at 100 Hz via a Bluetooth module (HC-06). A Bluetooth receiver is used to convert the digital signals from the DMWES into analyzable data for smartphones. The electronic fabric is led out from both ends of the interdigital electrode through copper tapes and then, connected to the PCB. The user 1's ECG signal is received by putting the electronic membrane on the chest. The generated ECG pulse signal is extracted from the pulse system and observed for ˜71 BPM, which proves the good healthy state of the user 1 (FIG. 21). These health parameters are of great significance in cardiovascular disease monitoring.

Moreover, the comparison between the moisture-wicking effect of the present invention and the commercial product after running exercise is made. FIG. 5G shows the optical and infrared sweating pictures of the electronic fabric before and after running exercise. When the electronic fabric is lifted up, it is observed that the skin beneath it is still dry. And the infrared picture demonstrates that the surface temperature of the electronic fabric is decreased about 1.8° C. after running because of the transferred moisture from the bottom hydrophobic layer, assuring the user's comfort and fast drying of inner layer, and signal stability. However, there is a lot of perspiration at the contact area between the commercial gel electrode and the skin, and it even fell off from the skin. The fabricated electronic fabric shows great application prospect in wearable electronic textiles with comfortable exercise experience.

In summary, the present invention provides an ultrasensitive directional moisture-wicking electronic fabric with dual-mode sensing capability and biomechanical energy harvesting based on the construction of heterogeneous fibrous membranes and the controllable MXene/CNTs electrospraying layer. Unidirectional moisture transfer is successfully realized by surface energy gradient and push-pull effect induced by the distinct hydrophobic-hydrophilic difference, which can spontaneously absorb sweat from the skin and ensure bioelectrical signal stability. The directional moisture-wicking electronic fabric shows superior static sensing properties, high sensitivity in low-pressure area, wide linear range, rapid response/recover time. In addition, the STENG based on the electronic fabric can deliver a high areal power density of 0 to 21.6 μW m⁻² and good cycling stability. With the superior pressure sensing and triboelectric performance, the electronic fabric can achieve all-range healthcare sensing, including accurate pulse monitoring, voice recognition, and gesture sensing. The present invention provides a electronic fabric and/or skin for the development for the next-generation breathable self-powered electronic skins in the applications of AI, human-machine interaction, and soft robots.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A biomimetic, nanofiber-based and directional moisture-wicking wearable electronic fabric having an asymmetric heterostructure, comprising:
a hydrophobic nanofiber layer;
a superhydrophilic nanofiber layer; and
a conductive layer;
wherein the conductive layer is located between the hydrophobic nanofiber layer and the superhydrophilic nanofiber layer, forming a multilayer composite structure to form a conductive and electronic sensing function and generate a surface energy gradient and a push-pull effect to guide moisture unidirectionally from a skin surface to an ambient atmosphere;
wherein the biomimetic, nanofiber-based and directional moisture-wicking electronic fabric has a pressure sensing sensitivity of 75 to 550 kPa⁻¹ in a pressure range from 0 to 20 kPa, response and recovery time of 28.4 ms and 39.1 ms, and a water vapor transfer rate between 12-18 kg·m⁻²·d⁻¹ at 25° C.

2. The biomimetic, nanofiber-based and directional moisture-wicking wearable electronic fabric of claim 1, wherein the conductive layer is coated on a side either of the hydrophobic nanofiber layer or the superhydrophilic nanofiber layer.

3. The biomimetic, nanofiber-based and directional moisture-wicking wearable electronic fabric of claim 1, wherein the hydrophobic nanofiber layer and the superhydrophilic nanofiber layer are electrospun nanofiber layers.

4. The biomimetic, nanofiber-based and directional moisture-wicking wearable electronic fabric of claim 1, wherein the conductive layer is an electrosprayed conductive layer.

5. The biomimetic, nanofiber-based and directional moisture-wicking wearable electronic fabric of claim 1, wherein the hydrophobic nanofiber layer comprises one or more high molecular weight hydrophobic polymers selected from polyvinylidene difluoride, copolymers of polyvinylidene difluoride, polyurethane, or polycaprolactone.

6. The biomimetic, nanofiber-based and directional moisture-wicking wearable electronic fabric of claim 1, wherein the superhydrophilic nanofiber layer comprises one or more high molecular weight hydrophilic polymer selected from polyacrylonitrile, polyvinyl alcohol, nylon or polyethylene glycol.

7. The biomimetic, nanofiber-based and directional moisture-wicking wearable electronic fabric of claim 1, wherein the conductive coating layer comprises:
an ultrafine and conductive functional material selected from one or more graphene oxide, titanium carbide, carbon nanotube, carbon black or acetylene black;
and a metal salt selected from lithium chloride or sodium chloride.

8. The biomimetic, nanofiber-based and directional moisture-wicking wearable electronic fabric of claim 7, wherein the size of the ultrafine and conductive functional material is 0.1 to 10 μm.

9. A method of fabricating the biomimetic, nanofiber-based and directional moisture-wicking wearable electronic fabric of claim 1, comprising:
forming either one of the hydrophobic nanofiber layer and the superhydrophilic layer by electrospinning;
electrospraying the conductive coating layer on a side of the hydrophobic or the superhydrophilic layer;
forming the other one of the hydrophobic nanofiber layer and the superhydrophilic layer on the conductive coating layer to form the multilayer composite structure with the conductive coating layer as the middle layer; and
drying them to obtain the biomimetic, nanofiber-based and directional moisture-wicking wearable electronic fabric.

10. The method of claim 9, wherein the electrospraying of the conductive coating layer comprises:
adding one or more ultrafine and conductive functional materials and a metal salt into a solution of water and ethanol with an addition of a surfactant to form an electrostatic spraying ink; and
electrospraying the electrostatic spraying ink on the side of the hydrophobic nanofiber layer or the hydrophilic nanofiber layer to form the conductive coating layer.

11. The method of claim 10, wherein the solution of water and ethanol has a mass ratio of (5-10): (10-20) of water and ethanol.

12. The method of claim 10, wherein the surfactant is a low molecular weight polyvinylpyrrolidone with an addition amount of 0-5 wt %.

13. The method of claim 10, wherein the ultrafine and conductive functional material selected from one or more graphene oxide, titanium carbide, carbon nanotube, carbon black or acetylene black; and the metal salt selected from lithium chloride or sodium chloride.

14. The method of claim 10, wherein the mass ratio among the one or more ultrafine and conductive functional materials, the surfactant and the solution is (2-5): (0-1): 20.

15. The method of claim 10, wherein the electrostatic spraying process is conducted by a single-needle electrospraying device with electrostatic spraying conditions of a voltage ranging from 18 kV to 25 kV, an injection pump flow rate of 0.04 mm/min to 0.1 mm/min, and a receiving distance between 5 cm to 15 cm.

16. The method of claim 9, wherein the formation of the hydrophobic nanofiber layer comprises:
adding a hydrophobic polymer and carboxylic carbon tubes to a dimethylformamide and/or acetone solvent and stirring them to obtain a first spinning solution; and
electrospinning the first spinning solution to obtain the hydrophobic nanofiber layer,
wherein the hydrophobic polymer is selected from polyvinylidene difluoride, copolymers of polyvinylidene difluoride, polyurethane, or polycaprolactone.

17. The method of claim 9, wherein the formation of the superhydrophilic nanofiber layer comprises:
adding a hydrophilic polymer and a surfactant to a dimethylformamide solvent and stirring them to obtain a second spinning solution; and
electrospinning the second spinning solution to obtain the superhydrophilic nanofiber layer,
wherein the hydrophilic polymer is selected from polyacrylonitrile, polyvinyl alcohol, nylon or polyethylene glycol and the surfactant is selected from sodium dodecyl sulfonate, sodium dodecyl benzene sulfonate or hexadecyl trimethyl ammonium bromide.

18. The method of claim 9, wherein the electrospinning is characterized by using a single-needle spinning device with electrospinning conditions comprising a voltage of 20 kV to 25 kV, a flow rate of syringe pump of 0.03 mm/min to 0.06 mm/min, and a receiving distance of 10 cm to 15 cm.

19. A biomimetic, nanofiber-based and directional moisture-wicking electronic skin, comprising the biomimetic, nanofiber-based and directional moisture-wicking wearable electronic fabric of claim 1, wherein the hydrophobic nanofiber layer is configured to contact human skin.

20. A single-electrode triboelectric nanogenerator, comprising the biomimetic, nanofiber-based and directional moisture-wicking wearable electronic fabric of claim 1, further comprising one or more electrodes positioned on the conductive layer, wherein the single-electrode triboelectric nanogenerator has an areal power density ranging from 0 to 21.6 μW m⁻².
